(19) United States
(12) Patent Application Publication
(10) Pub. No.: US 2019/0161798 A1
(43) Pub. Date: May 30, 2019

(54) Selection of Donors for Generation of Anti-Angiogenic Vaccine Compositions Including ValloVax

(71) Applicant: BATU BIOLOGICS, INC., San Diego, CA (US)

(72) Inventors: Samuel C. Wagner, San Diego, CA (US); Thomas E. Ichim, San Diego, CA (US)

(21) Appl. No.: 16/206,962

(22) Filed: Nov. 30, 2018

Related U.S. Application Data
(60) Provisional application No. 62/592,598, filed on Nov. 30, 2017.

(51) Int. Cl. C12Q 1/6881; C12Q 1/6804; C12N 5/073

(52) U.S. Cl. CPC C12Q 1/6881; C12N 2501/24; C12N 5/0605; C12Q 1/6804

(57) ABSTRACT

Disclosed are methods of selecting donors for production of anti-angiogenic vaccines in order to ensure maximal elicitation of immunity. In one embodiment, the invention teaches the purposeful mismatching of major and/or minor human leukocyte antigen (HLA) between the donor and recipient. In other embodiments the invention provides a system for generating a cell bank, said cell bank comprising different donor originals that are subsequently matched with recipients for optimal immune response. In another embodiment cells are transfected to induce immunogenicity, in some embodiments, transfected with allogeneic and/or syngeneic antigens.

### CROSS-REFERENCE TO RELATED APPLICATIONS

This Patent Application takes priority from Provisional Patent Application No. 62/592,598, titled Selection of Donors for Generation of Anti-Angiogenic Vaccine Compositions Including ValloVax, filed on Nov. 30, 2017, the contents of which are expressly incorporated herein by this reference as though set forth in their entirety and to which priority is claimed.

### BACKGROUND OF THE INVENTION

The invention belongs to the field of cancer immunotherapy. More particularly the invention belongs to the field of utilizing allogeneic cells for treatment of cancer by purposely mismatching donors and recipients of allogeneic immunogenic cells. More particularly, the invention teaches methods of mismatching immunogenic molecules from donor to recipient in order to maximize efficacy of allogeneic cancer vaccines, particularly cancer endothelial vaccines designed to inhibit proliferation and viability of tumor endothelium.

Tumor vaccines have been utilized for almost a century. Various successes and failures have been described in the literature, but one common feature is that in many cases there is a major degree of unpredictability of response.

Historically, one of the original papers describing the theoretical basis for cancer vaccines came. In 1943, when Ludwig Gross demonstrated in the C3H inbred mouse strain that rejection of tumor tissue would occur when transplanting a chemically induced sarcoma to a genetically identical mouse, in contrast to noncancerous tissue that would not be rejected. These tumor rejection experiments were repeated with similar results by several groups including Prehn and Main, Klein et al, and Old et al. Rejection of cancerous tissue in a syngeneic graft suggested the existence of tumor specific antigens that could be used as therapeutic targets. This sparked an interest in tumor immunology marked by many groups attempting to develop the “cancer vaccine.” The cancer vaccine is not aimed at inducing prophylaxis but to stimulate the otherwise dormant antitumor immune responses of the host. This response is referred to as “dormant” since an appropriate response would have eradicated the tumor. This dormancy must be overcome through “educating” the immune system that the tumor is part of nonself (or danger) and therefore needs to be eliminated. One way to educate the immune response to view the tumor as “danger” is to inject dead tumor cells into a site that is different from the site in which the tumor developed. Such an ectopic injection will present tumor antigens to the immune system in an anatomical location that is free from the local immunosuppressive effect of the growing tumor. To increase the probability of inducing an immune response, the dead cancer cells should be co-injected with an adjuvant, analogous to the way that bacterial vaccines provide the most protection when co-injected with adjuvant.

One of the most potent ways of inducing a “danger” signal is to immunize with cells of allogeneic origin. The anti-allogeneic immune response is one of the most potent immune responses. This is believed to be the case due to the high proportion of T cell receptors (TCR) recognizing allogeneic MHC. In a typical T cell activation program, antigen presenting cells present endogenous antigenic peptides on HLA I molecules and exogenous antigens on HLA II. HLA I presentation is mediated by practically all nucleated cells, whereas HLA II antigen presentation is restricted to antigen presenting cells such as B cells, monocytes, macrophages and dendritic cells. While regular antigen presentation occurs between the interaction of HLA with peptide inside the HLA molecule, allogeneic antigen presentation occurs with the TCR recognizing molecular motifs outside of the peptide binding groove, thus while peptide specific T cells are found approximately 1 in 100,000 to 1 in a million, allogeneic HLA recognizing T cells are found approximately in 1 out of 10 T cells. Thus, by immunizing with allogeneic tumor cells, the likelihood of stimulating immunity and indirect antigen presentation increases because of the large number of T cells attacking the allogeneic tumor, which results in cell death but more importantly, generation of immunogenic cytokines that stimulate antigen specific T cells.

Although multitudes of allogeneic cancer vaccination experiments were successful in animal models, when the “cancer vaccine” was tried in humans, very little, if any increase in patient survival was reported (reviewed in reference). The failure of this protocol was blamed in part on the unnaturalness of the models used in preclinical development. Specifically, the experimental models utilized to support tumor immunogenicity or to provide a basis for active clinical immunotherapy have been obtained from transplanted tumor systems which entail artefactual immunity associated with viral or chemical induction of the tumors or their allogeneic transplantation.

Cancer models were easily curable in mice and other small animals since the tumor cells used were highly immunogenic, this did not represent tumor cells found in the human population. For example, a type of tumor used to induce immunity as a preclinical model for tumor vaccination was the methylcholanthrene (MCA)-initiated neoplasm. MCA is a powerful carcinogen that induces neoplasia several weeks post administration. This type of cancer is not comparable to human cancers since one rarely develops cancer after a single large exposure to a carcinogen. The human cancer situation is a much more latent process that allows for the accumulation of several mutations over time. The significance of these mutations is that they allow for a variety of host evasion mechanisms to develop in the cancer, based on survival of the fittest cancer cell. The chemically induced cancer contains fewer mutations and has not undergone a natural selection process based on its immune evasion mechanisms. A demonstration of the immunogenicity of MCA induced cancers compared to spontaneously occurring cancers is seen when irradiated cells of each tumor are injected into syngeneic mice followed by a challenge with a live tumor inoculum of the same tumor used to vaccinate. Mice vaccinated with the immunogenic MCA tumors reject the inoculum whereas mice immunized with the nonimmunogenic spontaneous tumors succumb to neoplastic growth.

Another explanation for the clinical failure of the tumor vaccine is the immunosuppression that exists in patients entering clinical trials. Since many of them are in the terminal phase of their respective neoplasm, they are likely to possess very weakened immune function. In fact, anergy to a variety of antigens has been shown in several end-stage cancer patients. In addition to the immune suppression induced by the tumor, immunotherapy patients are routinely administered chemotherapy to reduce the initial tumor load; this further contributes to the state of immune suppression. Immunizing with tumor antigens when the patient is immunosuppressed may not only be ineffective, but even detrimental since it can lead to activation of a cancer promoting immune response. This response has been described by Prehn et al. Its existence has been substantiated by the T cell deficient nude mouse, which lacks a T-cell dependent immune system, and has a decreased rate of proliferation of transplanted sarcomas compared to control mice.

Despite this, some investigators reported survival increases. Mathe et al reported to use of irradiated allogeneic leukemic cells in 200 patients. In acute lymphatic leukemia, 57 out of 168 patients treated in this way remained in primary remission for 18 months to 10 years after active immunotherapy was begun, the relapse rate became low after 18 months and nil after 36 months. The results varied according to prognostic factors: the cytological type, active immunotherapy being above all effective in small cell (micro lymphoblastic and prolymphocytic) types with a hope of cure in 50 to 60 percent of cases; malignant cellular volume; meningeal deposits. In micro lymphoblastic forms the possibility of survival at the 5th year is greater than 90 percent. After relapse during active immunotherapy sensitivity to chemotherapy does not seem to be diminished. Trials of active immunotherapy in acute myeloid leukemia are worthy of further pursuit. The results of active immunotherapy in leukemic lymphosarcoma show that immunotherapy may be effective in preventing local recurrence, both of tumor as well as in the marrow. Four patients are in apparently complete remission for more than four years.

Further demonstration of efficacy signals using allogeneic vaccines come from studies in melanoma, where an immunization protocol was reported that consisted of the intradermal inoculation of 2 times 10(7) irradiated allogeneic melanoma cells admixed with 50 ug of percutaneous BCG. This method of immunization induced a significant but transient fall in the specific inhibitory effects of the sera on tumor directed cytotoxic activity of the patients' lymphocytes. In a clinical study 30 patients with disseminated malignant melanoma being treated with chemotherapy (DTIC and vincristine) the immunotherapy was given midway between courses of the cytotoxic drugs. There was a correlation between the effects on circulating inhibitor and clinical outcome. The number of objective regressions occurring in this small pilot group was surprisingly high ( 17/30).

Other studies did not show such positive responses. For example, in one publication, fifty-six patients with disseminated malignant melanoma were randomly allocated to two treatment groups. The first group C received combination chemotherapy consisting of DTIC and ICRF 159. The second group (C+I) received the same chemotherapy but were also immunized with 2 ×10(7) irradiated allogeneic melanoma cells mixed with 50 micrograms of percutaneous BCG. The survival rates in both treatment groups C and (C+I) were not significantly different, and only minor enhancement of the chemotherapy was found in the (C+I) group. A similar pattern of tissue response was observed in both groups: lymph node, skin and, to some extent liver metastases, respond better than other sites.

### DETAILED DESCRIPTION OF THE PREFERRED EMBODIMENTS

The invention overcomes the issue of heterogenic responses to allogeneic cellular immunization, particularly in the case of immunization with endothelial cells generated to evoke a response to tumor endothelial cells. The invention teaches means of matching donors to recipients so has to evoke the strongest possible allogeneic response.

In this invention, endothelial cells, and/or endothelial progenitor cells are collected from a genetically, racially and ethnically diverse population. After collection, the endothelial cells, and/or endothelial progenitor cells are processed so as to create a tumor endothelial specific cellular immunogen, typed and stored in units in a quick and cost-effective manner. Means of creating endothelial tumor specific cellular immunogens are known in the art and include ValloVax, which is developed by Batu Biologics and is incorporated by reference. The invention also is applicable to allogeneic cancer vaccines generated from allogeneic cancer cells or cell lines, including melanoma, lung, renal, prostate, pancreatic and leukemia based vaccines. The invention further covers the use of allogeneic gene transfected vaccines. Additionally, the invention envisions purposeful mis-matching for vaccines using allogeneic cellular lysate.

A record is provided for each unit, thereby creating an allogeneic stem cell bank. The units are then matched to unrelated individuals, not yet in need of vaccination, who have provided a biological sample. The matching units are available for the individual's use in case oncogenesis, the cells are collected from a genetically, racially and ethnically diverse population. After collection, the endothelial or endothelial progenitor cell products are processed, typed and stored in units in a quick and cost-effective manner. A record is provided for each unit, thereby creating an allogeneic cellular bank. The units are then matched to unrelated individuals, not yet in need of vaccination, who have provided a biological sample.

In one embodiment, the invention provides a method for providing an immunogenic endothelial or endothelial progenitor cell unit in an allogeneic cell bank for a potential recipient. This method includes the following. First, this method provides a plurality of immunogenic endothelial or endothelial progenitor cell units which have been typed.

The invention provides the use of tissue or circulating EPC as a substrate for transformation into an immunogenic cell population resembling tumor associated endothelial cells and subsequent use in a mis-matched manner. The EPC is an undifferentiated cell that can be induced to proliferate using the methods of the present invention. The EPC is capable of self-maintenance, such that with each cell division, at least one daughter cell will also be an EPC cell. EPCs are capable of being expanded 100, 250, 500, 1000, 2000, 3000, 4000, 5000 or more-fold. Phenotyping of EPCs reveals that these cells express the committed hematopoietic marker CD45. Additionally, an EPC is immunoreactive for VEGFR-2. The EPC is a multipotent progenitor cell. By multipotent progenitor cell is meant that the cell is capable of differentiating into more than one cell type. For example, the cell is capable of differentiating into an endothelial cell or a smooth muscle cell. Vascular endothelial growth factor (VEGF) acts through specific tyrosine kinase receptors that includes VEGFR-1 (flt-1) and VEGFR-2 (flk-1/KDR) and VEGFR-3/Flt-4 which convey signals that are essential for embryonic angiogenesis, cancer angiogenesis and hematopoiesis. While VEGF binds to all three receptors, most biological functions are mediated via VEGFR-2 and the role of VEGFR-1 is currently unknown. VEGFR3/Flt4 signaling is known to be important for the development of lymphatic endothelial cells and VEGFR3 signaling may confer lymphatic endothelial-like phenotypes to endothelial cells. VEGFRs relay signals for processes essential in stimulation of vessel growth, vasorelaxation, induction of vascular permeability, endothelial cell migration, proliferation and survival. Endothelial cells express all different VEGF-Rs. During embryogenesis, it has been reported that a single progenitor cell, the hemangioblast can give rise to both the hematopoietic and vascular systems. In the process of tumor angiogenesis, VEGF plays a fundamental role in promoting malignant and leaky angiogenesis.

The typed immunogenic endothelial or endothelial progenitor cell units of this invention form an allogeneic stem cell bank. Second, this method provides a record for each typed immunogenic endothelial or endothelial progenitor cell unit in the stem cell bank. Third, this method provides typing for a potential recipient of an immunogenic endothelial or endothelial progenitor cell unit and provides each potential recipient with a type identifier. Fourth, this method comprises storing the record for each typed immunogenic endothelial or endothelial progenitor cell and each type identifier in a database. Fifth, this method further comprises a comparison step whereby the type identifier is compared with each record for each typed immunogenic endothelial or endothelial progenitor cell unit to find a matched immunogenic endothelial or endothelial progenitor cell unit. And sixth, this invention provides a method for storing a matched immunogenic endothelial or endothelial progenitor cell unit in a database for a potential recipient's use, thereby providing an immunogenic endothelial or endothelial progenitor cell unit for a potential recipient. Preferably, the immunogenic endothelial or endothelial progenitor cell bank or depository or storage facility is a place where stem cells are kept for safe keeping.

In another embodiment, the present invention relates to a method for supplying an immunogenic endothelial or endothelial progenitor cell unit to an individual suffering from cancer, wherein said immunogenic endothelial or endothelial progenitor cell is purposely mismatched with prospective HLA typing of cancer patients. In one aspect, the invention provides a method for providing an immunogenic endothelial or endothelial progenitor cell unit in an allogeneic immunogenic endothelial or endothelial progenitor cell for many potential recipients.

In one specific embodiment of this invention, a recipient is treated with an immunogenic endothelial or endothelial progenitor cell unit, when for example, a potential recipient is suffering from cancer. In another aspect, many potential recipients suffer from cancer. In one aspect of the invention, the immunogenic endothelial or endothelial progenitor cell comprise ValloVax tumor endothelial cell vaccine.

In one embodiment of the invention, the immunogenic endothelial or endothelial progenitor cell.

The term “allogeneic” refers to cells, tissue, or organisms that are of different genetic constitution.

The term “type or “typing” as used herein refers to any and all characteristics of a sample, e.g., endothelial or endothelial progenitor cell product sample, which might be of relevance or importance for any potential use of the sample. The term and the corresponding testing conducted to determine the “type” of the sample is thus not limited to any particular tests mentioned herein, e.g., HLA typing. Determination of which tests are relevant and how to perform them is entirely conventional and will change with technological developments. Thus, the term “type identifier” refers to any characteristic that can be used for identification purposes.

The term “matching” refers to the degree of similarity between the genetic makeup of the cell product or unit to be used for vaccination into an individual and the individual's genetic makeup. For the purposes of this invention, when two people share a type, they are said to be a match meaning that their tissues are immunologically compatible with each other. The degree to which blood parameters need be identical will vary from patient to patient, and from year to year depending on the current state of technology. Matching then refers to providing the desired degree of match. For example, bone marrow and peripheral blood stem cell transplantation requires a greater degree of matching than blood cord stem cell transplantation. Matching can refer to a match with about 90%, 80%, 70%, 60%, 60%, or 40% similarity. A matching stem cell unit is one that is from a donor not related to the potential recipient.

The phrase “differentially present” refers to differences in the quantity or frequency (incidence of occurrence) of a marker present in a sample taken from a test subject as compared to a control subject. For example, a marker can be a gene expression product that is present at an elevated level or at a decreased level in blood samples of a risk subjects compared to samples from control subjects. Alternatively, a marker can be a gene expression product that is detected at a higher frequency or at a lower frequency in samples of blood from risk subjects compared to samples from control subjects.

A gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab') fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen encoded by a gene comprised in the genomic regions or affected by genetic. The immunoglobulin molecules of the invention can be of any type (e.g., IgG, IgE, IgM, IgD, IgA and IgY), class (e.g., IgG₁, IgG.sub.2, IgG.sub.3, IgG.sub.4, IgA.sub.1 and IgA.sub.2) or subclass of immunoglobulin molecule.

“Immunoassay” is an assay that uses an antibody to specifically bind an antigen (e.g., a marker). The immunoassay is characterized by the use of specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background. The phrase “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting said expression, in more general terms, intervening in said expression, for instance by affecting the expression of a gene encoding that protein.

In one embodiment, EPCs refer to endothelial colony-forming cells (ECFCs) and their progenitor cell capacities were characterized as described (Wu, Y et al., J Thromb Haemost, 2010; 8:185-193; Wang, H et al., Circulation research, 2004; 94:843 and Stellos, K et al., Eur Heart J., 2009; 30:584-593). Briefly, human blood was collected from healthy volunteer donors. All volunteers had no risk factors of CVD including hypertension, diabetes, smoking, positive family history of premature CVD and hypercholesterolemia, and were all free of wounds, ulcers, retinopathy, recent surgery, inflammatory, malignant diseases, and medications that may influence EPC kinetics. After dilution with HBSS (1:1), blood was overlaid onto Histopaque 1077 (Sigma-Aldrich Co. LLC, St. Louis, Mo.) in the ratio of 1:1 and centrifuged at 740 g for30 minutes at room temperature. Buffy coat MNCs were collected and centrifuged at 700 g for 10 minutes at room temperature. MNCs were cultured in collagen type I (BD Bioscience, San Diego) (50 m/ml)-coated dishes with EBM2 basal medium (Lonza Inc., Allendale, N.J.) plus standard EGM-2 SingleQuotes (Lonza Inc., Allendale, N.J.) that includes 2% fetal bovine serum (FBS), EGF (20 ng/ml), hydrocortisone (1 μg/ml), bovine brain extract (12 μg/ml), gentamycin (50 m/ml), amphotericin B (50 ng/ml), and epidermal growth factor (10 ng/ml). Colonies appeared between 5 and 22 days of culture were identified as a well-circumscribed monolayer of cobblestone-appearing cells. ECFCs with endothelial lineage markers expression, robust proliferative potential, colony-forming, and vessel-forming activity in vitro are defined as EPCs as described (Wang, H et al., Circulation research, 2004; 94:843 and Stellos, K et al., Eur Heart J., 2009; 30:584-593). Passage 4 to 6 EPCs were used for experiments. For a brief characterization, endothelial phagocytosis function was confirmed by incubating EPC in 4-well chamber slide with 1, 1-dioctadecyl-3, 3, 3, 3-tetramethylindocarbocyanine (DiI)-labeled acetylated low-density lipoprotein (acLDL) (Biomedical Technologies, Inc., Stoughton, Mass.) (5 m/ml) at 37° C. for 1 h, washed 3 times for 15 min in PBS, and then fixed with 2% paraformaldehyde for 10 min. Cells were then incubated with FITC conjugated UEA-1 (Ulex europaeus agglutinin) (10 m/ml) (Sigma-Aldrich Corporation, St. Louis, Mo.) for 1 h at room temperature, which is capable of binding with glycoproteins on the cell membrane to allow visualization of the entire cell. Cell integrity was examined by nuclear staining with DAPI (100 ng/ml). After staining, cells are imaged with high-power fields under an inverted fluorescent microscope (Axiovert 200, Carl Zeiss, Thornwood, N.Y.) at 200.times. magnification and quantified using Image J software.

In one aspect of the invention, the invention provides a computer-readable medium or combination of computer-readable media, containing a program for maintaining type information and providing a mismatched immunogenic endothelial or endothelial progenitor cell units for a potential recipient. This program contains code to affect the following. First, it provides a record of typed immunogenic endothelial or endothelial progenitor cell units in an allogeneic immunogenic endothelial or endothelial progenitor cell bank. Second, it provides a type identifier for a potential recipient. Third, it stores the type identifier and a record of typed immunogenic endothelial or endothelial progenitor cell units. Fourth, it compares the type identifier with the record of typed immunogenic endothelial or endothelial progenitor cell units to find a mis-matched immunogenic endothelial or endothelial progenitor cell unit. Fifth, it stores the mis-matched immunogenic endothelial or endothelial progenitor cell unit for the potential recipient's use. In one aspect of the invention, the medium or media of claim are selected from the group consisting of a RAM, a ROM, a disk, an ASIC, and a PROM.

The potential of using the tumor vasculature as a target is enticing, however previous studies have not utilized polyvalent antigenic entities, or in the cases where they have, such as in cellular vaccines, the cells where either not made to be immunogenic, nor are the cells grown under conditions which induce replicate the tumor microenvironment. The invention teaches augmentation of immunogenicity by purposely mis-matching donor and recipient. The following examples are provided to allow the practitioner of the invention to ascertain various immunization regimens, adjuvants, and combinations. The invention teaches means of “focusing” an immune response subsequent to immunization with a polyvalent cancer vaccine targeting tumor associated blood vessels. In one embodiment, patients suffering from cancer are immunized with ValloVax, or a vaccine composition similar to tumor endothelial cells. Active immunization against tumor endothelium by vaccinating against proliferating endothelium or markers found on tumor endothelium has provided promising preclinical data. Specifically, in animal models it has been reported that immunization to antigens specifically found on tumor vasculature can lead to tumor regression. Studies have been reported using the following antigens: survivin, endosialin, and xenogeneic FGF2R, VEGF, VEGF-R2, MMP-2, and endoglin. Human trials have been conducted utilizing human umbilical vein endothelial (HUVEC) cells as tumor antigens, with responses being reported in patients. In one report describing a 17-patient trial, Tanaka et al demonstrated that HUVEC vaccine therapy significantly prolonged tumor doubling time and inhibited tumor growth in patients with recurrent glioblastoma, inducing both cellular and humoral responses against the tumor vasculature without any adverse events or noticeable toxicities.

In one embodiment of this invention, the endothelial cell or endothelial progenitor cell products will be HLA typed. Standard techniques are known in the art for HLA typing, e.g., DNA typing or serological and cellular typing (Terasaki and McClleland, (1964) Nature, 204:998). One typing method for HLA identification purposes is restriction fragment length polymorphism analysis. Restriction fragment length polymorphism analysis relies upon the strong linkage between allele-specific nucleotide sequences within the exons that encode functionally significant HLA class II epitopes. Another method, PCR-SSO, relies upon the hybridization of PCR amplified products with sequence-specific oligonucleotide probes to distinguish between HLA alleles (Tiercy et al., (1990) Blood Review 4: 9-15, Saiki et al. (1989) Proc. Natl. Acad. Sci., U.S.A. 86: 6230-6234; Erlich et al. (1991) Eur. J Immunogenet. 18(1-2): 3355; Kawasaki et al. (1993) Methods Enzymol. 218:369-381). Yet another molecular typing method that can be used in the present invention, PCR-SSP, uses sequence specific primer amplification (Olerup and Zetterquist (1992) Tissue Antigens 39: 225-235). One of skill will also know how to type SSCP-Single-Stranded Conformational Polymorphism method. Other typing methods include high throughput methods of HLA typing. For example, one of skill will know how to amplify HLA sequences with allelic specific HLA primers and immobilize the amplification products to a solid surface using a labeled locus-specific or an allele-specific capture oligonucleotide. The presence of the oligonucleotides can then be detected and HLA allele analysis can be performed (U.S. application Ser. No. 09/747,391).

1. A method of selecting a donor for use of said donor cells in preparation of a therapeutic vaccine inhibiting recipient angiogenesis, the method comprising:
a) identifying antigenic determinants specific to said donor;
b) identifying antigenic determinants specific to said recipients; and
c) matching said recipient with said donor in a manner so as to allow for highest level of immunological reactivity between said donor cell and said recipient immune response.

2. The method of claim 1, wherein said donor cells are treated in a manner to augment immunogenicity by culture in interferon gamma at a concentration and time sufficient to increase expression of HLA antigens more than 50% as compared to baseline.

3. The method of claim 2, wherein said donor cells are placental endothelial progenitor cells that are extracted by a method selecting for fetal derived endothelial progenitor cells.

4. The method of claim 3, wherein less than 5% of said placental endothelial progenitor cells are of maternal origin.

5. The method of claim 3, wherein selection of fetal placental endothelial progenitor cells is accomplished through a method comprising:
(i) isolating a mammalian cellular population;
(ii) enriching for a subpopulation of the cells of step (i), which subpopulation expresses a CD45⁻ phenotypic profile;
(iii) enriching for a subpopulation of the CD45⁻ cells derived from step (ii) which express a CD34₊ phenotypic profile; and
(iv) isolating the subpopulation of CD34₊ cells derived from step (iii) which express a CD31^{lo/-} phenotypic profile, to thereby isolate the endothelial progenitor cells.

6. The method of claim 1, wherein said antigenic determinants are HLA alleles.

7. The method of claim 6, wherein said HLA allele is HLA-A.

8. The method of claim 6, wherein said HLA allele is HLA-B.

9. The method of claim 6, wherein said HLA allele is HLA-C.

10. The method of claim 6, wherein said HLA allele is HLA-DP.

11. The method of claim 6, wherein said HLA allele is HLA-DQ.

12. The method of claim 6, wherein said HLA allele is HLA-DR.

13. The method of claim 6, wherein said HLA allele is HLA-B27.

14. The method of claim 13, wherein said HLA allele is identified by antibodies.

15. The method of claim 13, wherein said HLA allele is identified by genotyping.
